(19) United States
(12) Patent Application Publication
(10) Pub. No.: US 2025/0352173 A1
(43) Pub. Date: Nov. 20, 2025

(54) ESTIMATION OF VISCOELASTICITY OF ARTERIAL OR VENOUS WALL

(71) Applicants: North Carolina State University, Raleigh, NC (US); MAYO FOUNDATION FOR MEDICAL EDUCATION AND RESEARCH, Rochester, MN (US)

(72) Inventors: Tuhin Roy, Raleigh, NC (US); Murthy Guddati, Raleigh, NC (US); Matthew W. Urban, Rochester, MN (US); Charles B. Capron, Rochester, MN (US)

(21) Appl. No.: 18/870,941

(22) PCT Filed: May 30, 2023
(86) PCT No.: PCT/US2023/067619

§ 371 Date: Dec. 2, 2024

Related U.S. Application Data
(60) Provisional application No. 63/346,983, filed on May 30, 2022.
(60) Provisional application No. 63/411,924, filed on Sep. 30, 2022.

(51) Int. Cl. A61B 8/08; A61B 8/00

(52) U.S. Cl. CPC A61B 8/0891; A61B 8/485

(57) ABSTRACT

Various examples are provided related to estimation of viscoelasticity of an arterial or venous wall. In one example, a method includes obtaining ultrasound data of an arterial or venous wall for a defined acoustic radiation force; and determining viscoelasticity of the arterial or venous wall. The viscoelasticity can be determined based upon correlation between measured and simulated wall velocity of the ultrasound data in a space-time, a wavenumber-frequency, wavenumber-time, or space-frequency domain; or in a sequential manner by determining the elastic part of the modulus by matching measured and simulated phase velocities and determining the viscoelasticity part by matching measured and simulated wall velocities; or a combination of both. The simulated velocity can be determined for a wall thickness and viscoelastic modulus of the arterial or venous wall through full wave analysis. In another example, a system includes an ultrasound scanner and a computing device that can implement the method.

### CROSS REFERENCE TO RELATED APPLICATIONS

This application claims priority to, and the benefit of, co-pending U.S. provisional application entitled “Method to Estimate Viscoelasticity of Arterial Wall” having Ser. No. 63/346,983, filed May 30, 2022, and co-pending U.S. provisional application entitled “Group-Velocity Based Approach to Estimate the Viscoelasticity of Arterial or Venous Wall” having Ser. No. 63/411,924, filed Sep. 30, 2022, which are both hereby incorporated by reference in their entireties.

### STATEMENT REGARDING FEDERALLY SPONSORED RESEARCH OR DEVELOPMENT

This invention was made with government support under grant number CMMI1635291 awarded by the National Science Foundation and grant number HL145268 awarded by the National Institutes of Health. The government has certain rights in the invention.

### BACKGROUND

According to World Health Organization, an estimated 17.9 million people die globally each year from cardiovascular diseases, an estimated 32% of all deaths worldwide. To detect cardiovascular diseases at an early stage, an important biomarker is arterial stiffness which is affected by both elastic modulus and viscosity. While there exist many methods to estimate elastic modulus, there does not seem to exist a reliable method to estimate complete viscoelasticity, i.e., the elastic and viscous parts of the modulus.

### SUMMARY

Aspects of the present disclosure are related to estimation of viscoelasticity of an arterial or venous wall. In one aspect, among others, a method for estimation of viscoelasticity of an arterial or venous wall comprises obtaining ultrasound data of an arterial or venous wall for a defined acoustic radiation force (ARF); and determining viscoelasticity of the arterial or venous wall. The viscoelasticity can be determined based upon correlation between measured and simulated wall velocity of the ultrasound data in a space-time, a wavenumber-frequency, wavenumber-time, or space-frequency domain, where the simulated velocity is determined for a wall thickness and viscoelastic modulus of the arterial or venous wall through full wave analysis; or in a sequential manner by determining the elastic part of the modulus by matching measured and simulated group or phase velocities and determining the viscous part by matching measured and simulated decay characteristics; or using a combination of both. Determination of the viscoelastic modulus can be based upon maximization of a correlation coefficient to within a defined threshold, in the space-time, wavenumber-frequency, wavenumber-time, or space-frequency domain.

In one or more aspects, the method can comprise matching measured phase velocity of the ultrasound data with simulated phase velocity corresponding to wave modes of the arterial or venous wall. The measured phase velocity can be matched with simulated phase velocity to within a defined threshold. The wave modes can comprise first and second circumferential modes. In various aspects, a measured decay profile can be matched with simulated decay profile to within a defined threshold. The method can comprise determining shear modulus prior to viscoelasticity determination based upon shear wave elastography (SWE) measurements. The SWE measurements can comprise local lumen radius (r), wall thickness (h) and induced wave group velocity (C_{g}). The shear modulus can be determined using an interpolation matrix. The interpolation matrix can be generated based at least in part upon acoustic radiation force push spatial and temporal characteristics. The interpolation matrix can be generated based upon local lumen radius (r), wall thickness (h) and induced wave group velocity (C_{g}).

In another aspect, a system for estimation of viscoelasticity of an arterial or venous wall comprises an ultrasound scanner configured for shear wave elastography (SWE) and a computing device comprising a processor and memory. The computing device can be configured to at least obtain ultrasound data of an arterial or venous wall for a defined acoustic radiation force (ARF), the ultrasound data obtained via the ultrasound scanner; and determine viscoelasticity of the arterial or venous wall. The viscoelasticity can be determined based upon correlation between measured and simulated wall velocity of the ultrasound data in a space-time, a wavenumber-frequency, wavenumber-time, or space-frequency domain, where the simulated velocity is determined for a wall thickness and viscoelastic modulus of the arterial or venous wall through full wave analysis; or in a sequential manner by determining the elastic part of the modulus by matching measured and simulated group or phase velocities and determining the viscoelasticity part by matching measured and simulated wall velocities; or using a combination of both. Determination of the viscoelastic modulus can be based upon maximization of a correlation coefficient to within a defined threshold, in the space-time, wavenumber-frequency, wavenumber-time, or space-frequency domain.

In one or more aspects, matching measured and simulated phase velocities can comprise matching measured phase velocity of the ultrasound data with simulated phase velocity corresponding to wave modes of the arterial or venous wall. The measured phase velocity can be matched with simulated phase velocity to within a defined threshold. The wave modes can comprise first and second circumferential modes. A measured decay profile can be matched with simulated decay profile to within a defined threshold. In various aspects, shear modulus can be determined prior to viscoelasticity determination based upon shear wave elastography (SWE) measurements. The SWE measurements can comprise local lumen radius (r), wall thickness (h) and induced wave group velocity (C_{g}). The shear modulus can be determined using an interpolation matrix. The interpolation matrix can be generated based at least in part upon acoustic radiation force push spatial and temporal characteristics. The interpolation matrix can be generated based upon local lumen radius (r), wall thickness (h) and induced wave group velocity (C_{g}). The ultrasound data can be obtained from the ultrasound scanner in real time or near real time or can be obtained from memory after recording by the ultrasound scanner.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of the geometry of the immersed axisymmetric tube which mimics a healthy human carotid artery, in accordance with various embodiments of the present disclosure.

FIG. 2 illustrates an example of simulated phase velocity dispersion curves corresponding to different wave modes in accordance with various embodiments of the present disclosure.

FIGS. 3A-3C illustrate an example of an applied excitation force in axial and circumferential directions, and in time, in accordance with various embodiments of the present disclosure.

FIG. 4 illustrates an example of generated full wave data with the considered excitation force, geometric and material properties, in accordance with various embodiments of the present disclosure.

FIGS. 5A-5C illustrates examples of full measured data, the corresponding k-t domain data, and the finally matching the k-t domain data with the damped single degree of freedom system at specific k ranges, in accordance with various embodiments of the present disclosure.

FIG. 6 illustrates an example of the estimated decay rate as a function of wavenumber, in accordance with various embodiments of the present disclosure.

FIGS. 7A and 7B illustrate examples of the effect of the modulus keeping the relaxation time at 0.055 ms, and effect of the relaxation time keeping the modulus at 200 kPa on the phase velocity dispersion, in accordance with various embodiments of the present disclosure.

FIGS. 8A and 8B illustrate examples of the effect of elastic modulus maintaining the damping ratio at 0.15 and effect of damping ratio maintaining the elastic modulus at 250 kPa, on the k-t domain decay rate (Approach 2), in accordance with various embodiments of the present disclosure.

FIGS. 9 and 10 illustrate examples of the effect of modulus keeping the relaxation time at 0.055 ms and effect of relaxation time keeping the modulus parameter of 200 kPa on full-wave response, in accordance with various embodiments of the present disclosure.

FIGS. 11 and 12 illustrate examples of the effect of modulus keeping the relaxation time at 0.055 ms and effect of relaxation time keeping the modulus parameter of 200 kPa on k-w response of full-wave data, in accordance with various embodiments of the present disclosure.

FIGS. 13A and 13B illustrate examples of synthetic data with noise added at a medium noise level and a higher noise level, in accordance with various embodiments of the present disclosure. SNR stands for signal-to-noise ratio.

FIGS. 14A and 14B illustrate examples of objective function variation with Approach 3 for Kelvin-Voigt viscoelastic model for synthetic data: (a) with medium noise; (b) with higher noise, in accordance with various embodiments of the present disclosure. The white asterisk is the point of the minimum objective function value.

FIGS. 15A and 15B illustrate examples of Iteration history of the gradient optimization for Kelvin-Voigt viscoelastic model for synthetic data with (a) medium noise and (b) higher noise, in accordance with various embodiments of the present disclosure.

FIGS. 16A and 16B illustrate examples of Noise-laden Synthetic data with spring-pot model: (a): medium noise level; (b): higher noise level, in accordance with various embodiments of the present disclosure. SNR stands for signal-to-noise ratio.

FIGS. 17A and 17B illustrate examples of objective function variation with Approach 3 for the spring-pot viscoelastic model for synthetic data: (a) with medium noise; (b) with higher noise, in accordance with various embodiments of the present disclosure. The white asterisk is the point of the minimum objective function value.

FIGS. 18A and 18B illustrate examples of iteration history of the gradient optimization for spring-pot viscoelastic model for synthetic data with (a) medium noise and (b) higher noise, in accordance with various embodiments of the present disclosure.

FIGS. 19A and 19B illustrate examples of objective function variation with Approach 4 for Kelvin-Voigt viscoelastic model for synthetic data: (a) with medium noise; (b) with higher noise (b), in accordance with various embodiments of the present disclosure. The white asterisk is the point of the minimum objective function value which coincides with the value that was used to get the synthetic data.

FIGS. 20A and 20B illustrate examples of objective function variation with Approach 4 for Spring-pot viscoelastic model for synthetic data: (a) with medium noise; (b) with higher noise, in accordance with various embodiments of the present disclosure. The white asterisk is the point of the minimum objective function value which coincides with the value that was used to get the synthetic data.

FIG. 21 illustrates an example of a process for performing shear modulus estimation, in accordance with various embodiments of the present disclosure.

FIGS. 22A and 22B illustrate examples of Semi-Analytical Finite Element (SAFE) simulation and an interpolation matrix populated with simulation results, in accordance with various embodiments of the present disclosure.

FIGS. 23A and 23B illustrates examples of geometry-dependent shear modulus underestimation introduced by using the bulk wave approximation and ameliorated based on the SAFE simulation and interpolation-based approach, in accordance with various embodiments of the present disclosure.

FIG. 24 is a schematic block diagram illustrating an example of a system employed for estimation of viscoelasticity of an arterial wall, in accordance with various embodiments of the present disclosure.

### DETAILED DESCRIPTION

Disclosed herein are various examples related to estimation of viscoelasticity of an arterial or venous wall. Reference will now be made in detail to the description of the embodiments as illustrated in the drawings, wherein like reference numbers indicate like parts throughout the several views.

Shear Wave Elastography (SWE) is a general technique used by biomedical engineers to characterize tissues based on propagation characteristics of the shear waves. A technique has been developed at Mayo Clinic, among others, to generate these shear waves using focused ultrasound, which is referred to as acoustic radiation force (ARF). ARF-SWE, which is an active research technique, has been used to characterize various tissues. ARF-SWE data obtained from arteries can be used to estimate the elasticity and viscosity (viscoelasticity) of the arterial walls, which is considered a biomarker for early onset of many cardiovascular diseases. A computationally efficient simulation of ARF-generated shear wave propagation through arterial wall and parameter estimation technique that estimates wall viscoelasticity by maximizing the correlation between the measured and simulated wall motion is presented. The methodology has been verified using in silico data (noise-laden synthetic data) and can be validated using phantom and ex vivo data.

Arterial stiffness is one of the important biomarkers for many cardiovascular diseases. To estimate the arterial stiffness non-invasively, the standard technique is to consider the Pulse Wave Velocity. However, the Pulse Wave Velocity approach suffers many limitations, including the global nature of the measurement. In contrast, the local arterial stiffness measurements can be performed using Acoustic Radiation Force (ARF) based imaging. In the ARF setting, an ultrasound wave propagates through the tissue material and the tissue and is absorbed by the tissue. The momentum of the ultrasound wave is transferred to the medium to generate tissue motion, response is analyzed spatially and temporally for characterizing the tissue locally. There are several ARF methods, among which Shear Wave Elastography (SWE) has become a promising tool. In the case of organs with confined geometry such as artery, the shear wave becomes guided and dispersive (phase velocity changes with frequency). Specifically, the arterial wall motion data is processed to obtain the phase-velocity dispersion, which is then used to invert for arterial wall modulus. While this approach works well for estimating the elasticity part of the arterial wall modulus, it fails to quantify viscosity, as the phase velocity dispersion curves are not sensitive with the arterial viscosity.

In the SWE setting, there are many waveguide models such as an immersed plate model, annuli waveguide, hollow tube waveguide, fluid-filled tube, immersed fluid-filled 3D finite element model, immersed fluid-filled SAFE model. These models can be used to estimate modulus from wave propagation measurements. The immersed plate model assessed the effect of both elasticity and viscoelasticity on the phase velocity dispersion, and it was concluded that the phase velocity dispersions are not influenced by the viscosity. However, wall viscosity is often considered an important biomarker in addition to stiffness. For the bulk organs (where the shear wave propagation is not affected by the organ boundaries), there are several approaches based on Rheological model and model free approaches. Two such models are Attenuation Measuring Ultrasound Shearwave Elastography, AMUSE, and the Two-point Frequency Shift method. While these approaches work well for the bulk organs, they are not effective in the case of arteries due to geometric confinement drastically altering both wave speed and attenuation.

In this disclosure, four approaches to estimate viscoelastic shear moduli are evaluated: Approach 1 considers the phase velocity dispersion in the wavenumber-frequency domain; Approach 2 utilizes at the decay rate of the wavenumber-time domain; Approach 3 tries to match the simulated and observed motion directly in space-time domain; Approach 4 attempts to match the simulated and observed motion in wavenumber-frequency domain. Based on analyses of the four approaches, a hybrid method combining Approaches 1 and 2 or, alternatively, Approach 3 or Approach 4 can be used to estimate the viscoelastic shear-modulus, which includes both storage and loss moduli. At this point, the proposed approaches were verified by applying to noise-laden synthetic data, leading to the conclusion that the Approach 4 is recommended to estimate the arterial viscoelasticity.

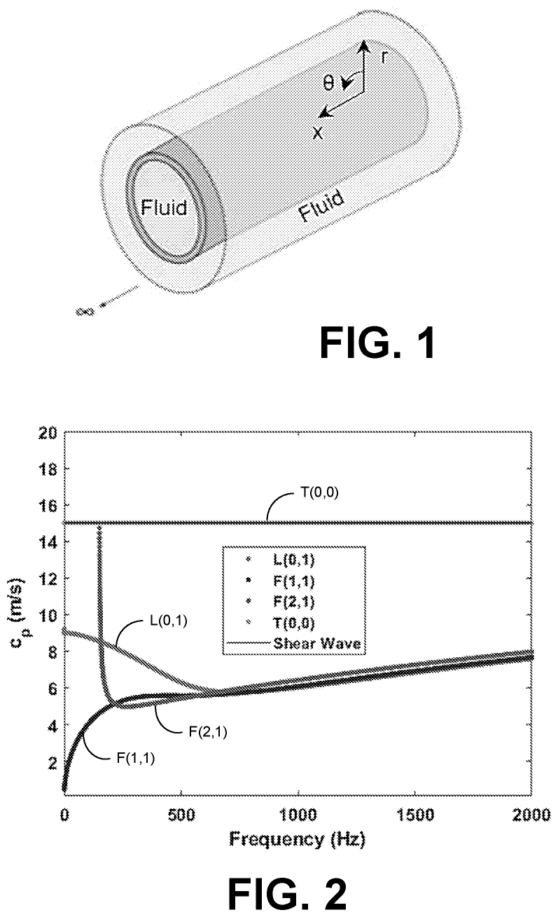

Given the arterial (or venous) wall velocity measurements, the objective is to estimate the arterial (or venous) elasticity and viscosity, essentially the viscoelastic shear modulus. For this, the carotid artery can be modeled as an axisymmetric incompressible tube. The blood in the artery as well as the surrounding tissue can be considered as inviscid fluids given that the shear wave speeds in these domains are negligible compared to the arterial wall. The schematic of the problem is shown in FIG. 1, which illustrates the geometry of the immersed axisymmetric tube which mimics health human carotid artery.

The motion of the solid domain (Ω_{S}) can be represented by the Elastodynamic equation,

$${L}_{σ}^{T} σ + ρ \frac{{\partial }^{2} u}{\partial {t}^{2}} = f , in {Ω}_{S} . \qquad ( 1 )$$

The incompressible fluid domain (Ω_{F}) is governed by the Laplace equation,

$${∇}^{2} p = 0 in {Ω}_{F} . \qquad ( 2 )$$

The arterial wall motion is coupled to the interior and exterior fluid responses through interface conditions at the solid-fluid domain interface (Γ_{SF}) representing the continuity of velocity and traction,

$$σ \cdot {n}_{s} - {pn}_{F} = 0 , on {Γ}_{SF} , \qquad ( 3 )$$

$$\frac{{\partial }^{2} u}{\partial {t}^{2}} \cdot {n}_{s} - \frac{1}{{ρ}_{F}} \frac{\partial p}{\partial {n}_{F}} = 0 , on {Γ}_{SF} . \qquad ( 4 )$$

For the solid medium, the primary variable is the displacement vector, u=u(r,θ,z,t) with 3 components namely u={uᵣ,u_{θ},u_{z}}^{T}. The stress tensor is σ=λ*tr(ò)I +2G*ò, where ò is the strain tensor (written in vector form) as ò=L_{ò}u={òᵣᵣ,ò_{θθ},ò_{zz},ò_{θz},ò_{rz},ò_{rθ}}^{T}. The symbol, * is the convolution operator. In the limit of incompressibility, the Lamé operator λ approaches infinity, while the shear modulus operator G is finite. For a Kelvin-Voigt model, the shear modulus can be formally written in an operator form,

$$G ( t ) = {G}_{0} ( 1 + τ \frac{\partial }{\partial t} ) , \qquad ( 5 )$$

where G₀ is the modulus parameter and τ is the relaxation time. For the spring-pot model, the shear modulus is an integro-differential operator, written in an abstract operator form as,

$$G ( t ) = {{G}_{0} ( \frac{1}{{ω}_{0}} \frac{\partial }{\partial t} )}^{α} , \qquad ( 6 )$$

where G₀ is the modulus factor and α is the fractional order (note that G₀ in Equations (5) and (6) are different). ω₀ is the reference angular frequency, which can be chosen arbitrarily; the only purpose is to maintain dimensional consistency of G₀ in Equations (5) and (6) (chosen as 600 Hz in the study, to be consistent with chosen frequency range).

The acoustic radiation force f=f(r,θ,z,t). The density of the solid medium is ρ. The 6×3 gradient operators, L_{σ} and L_{ò} can be found in “Shear wave dispersion analysis of incompressible waveguides” by Roy et al. (J. Acoust. Soc. Am. 149 972-82 (2021)). For the fluid medium, the primary variable is the pressure p=p(r,θ,z,ω). The Laplace operator in cylindrical coordinate system is given by, ∇²(·)=r⁻¹∂(r∂(·)/∂r)/∂r+r⁻²∂²(·)/∂θ²+∂²(·)/∂z². In the interface conditions, nₛ and n_{F} are the unit vectors for solid and fluid domain respectively (opposite vectors), and ρ_{F} is the fluid density.

### SAFE and PMDL Framework

Due to the invariant geometry and material properties along the axial and circumferential direction, the Semi-Analytical Finite Element (SAFE) framework can be utilized. Specifically, the harmonic expansion is used in temporal, axial and circumferential directions, while the finite element discretization can be applied along the radial direction. Therefore, for each of the wave modes, the solutions take the form,

$$u ( r , θ , x , t ) = {{\int }^{ω = + ∝}}_{ω = - ∝} {{\int }^{k = + ∞}}_{k = - ∞} {{\int }^{m = ∞}}_{m = 0} {N}_{s} ( r ) [ \begin{matrix}{U}_{r} ( m , k , ω ) cos ( m θ ) \\ {U}_{θ} ( m , k , ω ) sin ( m θ ) \\ {U}_{z} ( m , k , ω ) cos ( m θ )\end{matrix} ] {e}^{- ikx} {e}^{i ω t} dt d ω , \qquad ( 7 )$$

$$p ( r , θ , x , t ) = {{\int }_{ω = - ∞}}^{ω = + ∞} {{\int }^{k = + ∞}}_{k = - ∞} {{\int }^{m = ∞}}_{m = 0} {N}_{F} ( r ) P ( m , k , ω ) cos ( m θ ) {e}^{- ikx} {e}^{i ω t} dk d ω , \qquad ( 8 )$$

where N_{S} and N_{F} are the finite element shape functions along the radial direction for the solid and fluid domain respectively, m is the index of the azimuthal harmonic, k is the wavenumber along the axial direction, ω=2πf is the temporal frequency, and i=\sqrt{−1}. Substituting equations (7) and (8) in governing equations (1) to (4), the discretized system gives:

$$( K - {ω}^{2} M ) \{ \begin{matrix}U \\ P\end{matrix} \} = \{ \begin{matrix}F \\ 0\end{matrix} \} , \qquad ( 9 )$$

$$where ,$$

$$K = [ \begin{matrix}{k}^{2} {K}^{S 2} + ik {K}^{S 1} + {K}^{S 0} & - κ {C}_{SF} \\ 0 & κ ( {k}^{2} {K}^{F 2} - {K}^{F 0} )\end{matrix} ] , M = [ \begin{matrix}{M}^{S} & 0 \\ {C}_{SF}^{T} & 0\end{matrix} ] . \qquad ( 10 )$$

[symbol]

is the normalization factor to improve the conditioning of the system. The solid-domain contribution matrices, K^{S2}, K^{S1}, K^{S0}, M^{S}, the fluid-domain contribution matrices, K^{F2}, K^{F0}, and the fluid-structure interaction matrix, C_{SF} are defined in “Dispersion analysis of composite acousto-elastic waveguides” by Vaziri Astaneh et al. (Compos. Part B Eng. 130 200-16 (2017)). These contribution matrices depend on the geometry (inner radius and thickness) and the material properties (densities and shear modulus). The Fₙₘ is Fourier transform of the applied forcing f(r,θ,z,t). Similarly, Uₙₘ and Pₙₘ are the Fourier transforms of primary variables u and p, respectively.

The radial discretization, N_{S} and N_{F} includes linear finite elements for the solid and interior fluid domains, and Perfectly Matched Discrete Layers (PMDL) for the surrounding fluid. To address the volumetric locking, selective reduced integration scheme can be utilized. The above discretized system can be analyzed in two ways which are discussed in the following subsections.

### Dispersion Relation Through k(ω) Analysis

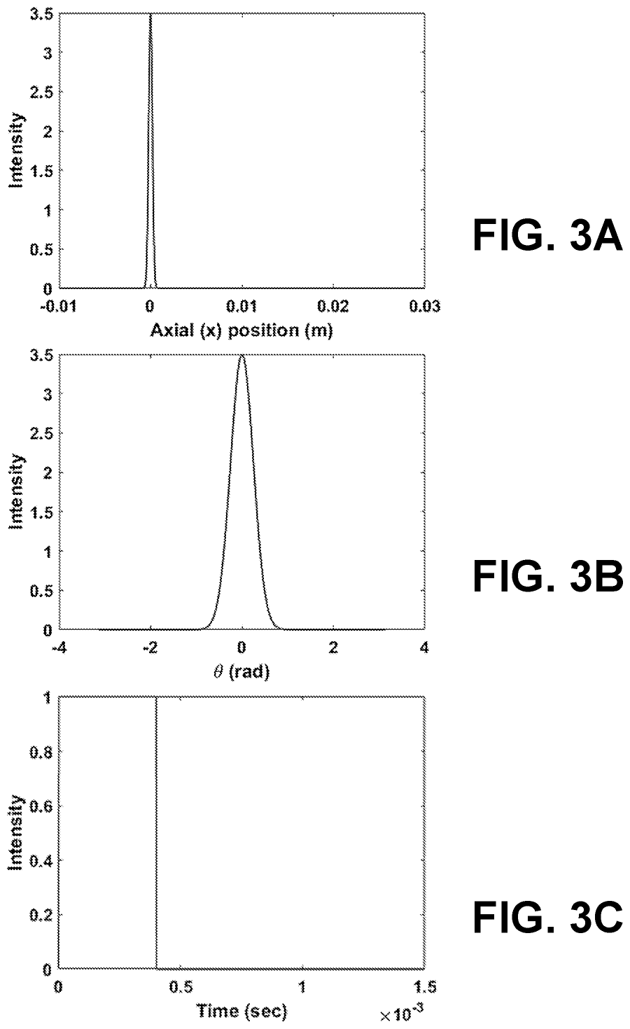

To get the dispersion relation, modal analysis of the above dynamical system (equation (9)) can be performed for given angular frequency, ω. Specifically, the following quadratic Eigenvalue problem can be solved for the unknown kₘ corresponding to m^{th} azimuthal harmonic:

$$[ {k}_{m}^{2} [ \begin{matrix}{K}^{S 2} & 0 \\ 0 & κ {K}^{F 2}\end{matrix} ] + {ik}_{m} [ \begin{matrix}{K}^{S 1} & 0 \\ 0 & 0\end{matrix} ] + [ \begin{matrix}{K}^{S 0} - {ω}^{2} {M}^{S} & - κ {C}_{SF} \\ - {ω}^{2} {C}_{SF}^{T} & κ {K}^{F 0}\end{matrix} ] ] \{ \begin{matrix}{ϕ}_{m}^{S} \\ {ϕ}_{m}^{F}\end{matrix} \} = \{ \begin{matrix}0 \\ 0\end{matrix} \} , \qquad ( 11 )$$

where ϕ^{S} and ϕ^{F} are the mode shapes corresponding to the displacements and pressure in solid and fluid domains, respectively. This quadratic Eigenvalue problem can be transformed to a linear Eigenvalue problem by rearranging the degrees of freedom (see “Improved inversion algorithms for near-surface characterization” by Vaziri Astaneh A and Guddati M N (Geophys. J. Int. 206 1410-23 2016) for details). Once the Eigenvalues are obtained, consider the lowest real wavenumbers, k₁ₘ and compute the phase velocities as c_{p,1m}=ω/k₁ₘ. Then plot the phase velocities as a function of cyclic frequency (Hz) as shown in FIG. 2.

### Full-Wave Simulation Through ω(k) analysis

To get the full wave simulation from the equation (9), For a given k, Equation (9) can be solved using modal analysis approach; the smoothness of the response across the arterial wall thickness helps a few initial modes to almost capture the full dynamics of the system. Specifically, Equation (9) can be decoupled using modal analysis by first solving the associated eigenvalue problem:

$$[ [ \begin{matrix}{k}^{2} {K}^{S 2} + ik {K}^{S 1} + {K}^{S 0} & - κ {C}_{SF} \\ 0 & κ ( {k}^{2} {K}^{F 2} - {K}^{F 0} )\end{matrix} ] - {ω}^{2} [ \begin{matrix}{M}^{S} & 0 \\ {C}_{SF}^{T} & 0\end{matrix} ] ] \{ \begin{matrix} \\ \end{matrix} \} = \{ \begin{matrix}0 \\ 0\end{matrix} \} , \qquad ( 12 )$$

Note that here ω are the resonant (free-vibration) frequencies associated with the chosen wavenumber k. [symbol] and [symbol] are the mode shapes for the solid and fluid domain respectively. The response is then written using modal superposition:

$$\{ \begin{matrix}U ( k , ω ) \\ P ( k , ω )\end{matrix} \} = {\sum }_{i = 1}^{N} {γ}_{i} ( ω ) \{ \begin{matrix} \\ \end{matrix} \} , \qquad ( 13 )$$

where γᵢ is the modal participation factor, and N is the total number of normal modes considered in the (truncated) modal expansion. Substitution of Equation (13) in Equation (9), leads to:

$${k}_{i} {γ}_{i} - {ω}^{2} {m}_{i} {γ}_{i} = {f}_{i} \qquad ( 14 )$$

which represents the vibration of a single-degree-of-freedom system in frequency domain, which can be analyzed more efficiently than the original system in Equation (9). Here, kᵢ=ϕₗ^{T}Kϕᵢ, mₗ=ϕᵢ^{T}Mϕᵢ, and fᵢ=ϕᵢ^{T}F are the modal stiffness, mass, and force respectively, where, ϕᵢ={\tilde{ϕ}^{S} \tilde{ϕ}^{F}} and K, M are defined in Equation (10). Equation (14) can be solved using frequency-response-function (in frequency domain) or impulse-response-function (in the time domain) formalisms depending on the employed viscoelasticity model. Further details can be found in “Full waveform inversion for arterial viscoelasticity” by Roy, T. and Guddati, M. N. (Physics in Medicine & Biology, 68(5), p. 05NT02 (2023)).

The complete response in wavenumber-time (k-t) domain is computed by first solving Equation (14) for multiple values of normal modes i and circumferential Fourier numbers m, and performing superposition:

$$\overline{U} ( k , t ) = {\sum }_{m = 0}^{M} {\sum }_{i = 1}^{N} {\overline{γ}}_{i , m} ( t ) {( {\tilde{ϕ}}_{i , m} ) [ cos ( m θ ) , sin ( m θ ) , cos ( m θ ) ]}^{T} . \qquad ( 15 )$$

The final space-time (x-t) can then be obtained through inverse Fourier transformation:

$$u ( x , t ) = {\int }_{- ∞}^{∞} \overline{U} ( k , t ) {e}^{2 π ikx} dk \qquad ( 16 )$$

While this approach can yield the response in 3D space and time, for the sake of computational efficiency, the computation can be limited to the radial response at the top wall.

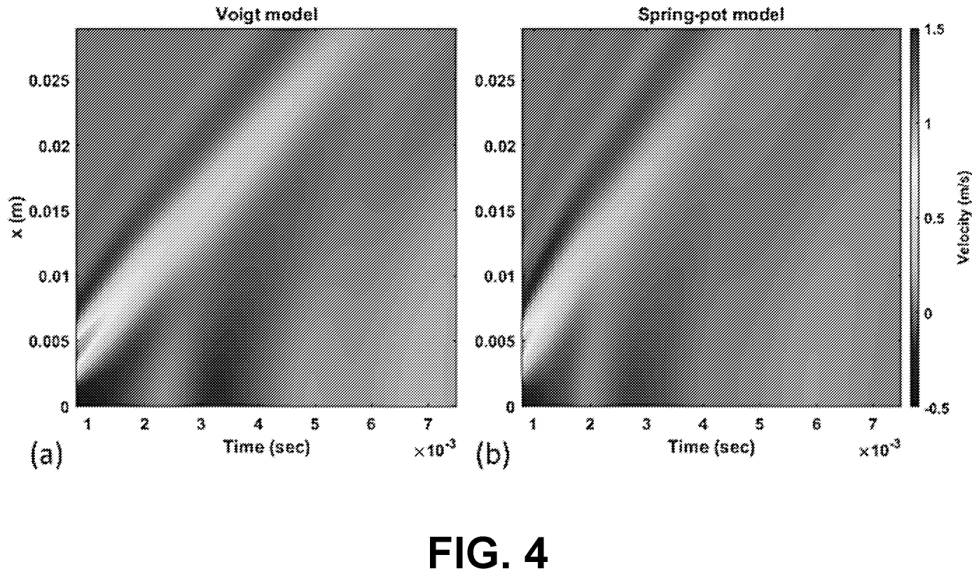

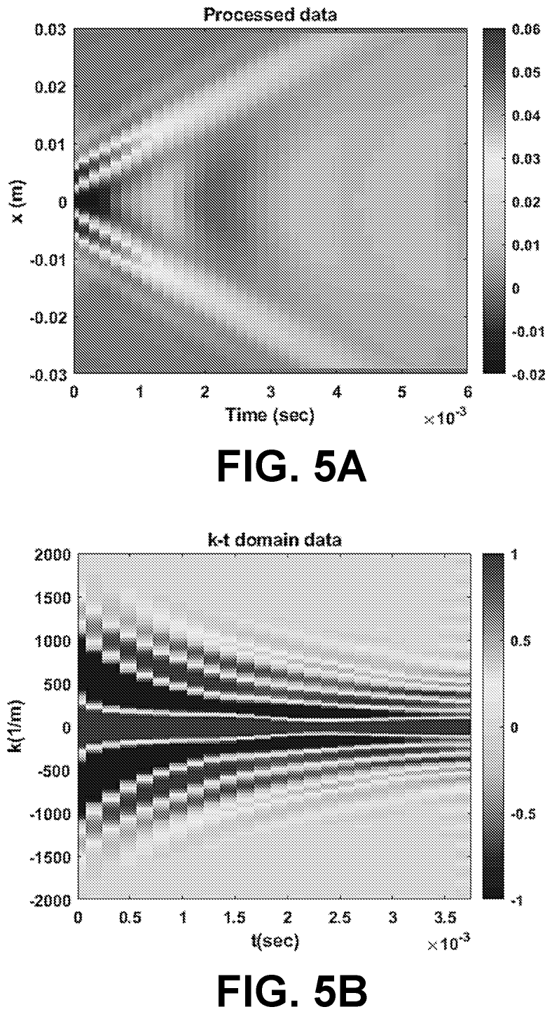

Guided by the artery mimicking phantoms considered in “Multimodal guided wave inversion for arterial stiffness: methodology and validation in phantoms” by Roy et al. (Phys. Med. Biol. 66 115020 (2021)), consider a tube with an inner radius of 3 mm and a wall thickness of 1 mm. The solid domain density is taken as the typical value of 1000 kg/m³. With respect to the viscoelasticity, consider two models, Kelvin-Voigt, and spring-pot. For the Kelvin-Voigt model, consider an elastic modulus of 200 kPa and a relaxation time of 0.055 ms. In the case of Spring-pot model, the modulus parameter is 300 kPa and the fractional order is 0.15. The interstitial and surrounding fluid is taken as water. The forcing function is assumed to be a Gaussian with a spread of 0.2 mm in the axial (x) direction and 0.25 radians in the circumferential (θ) direction (see FIGS. 3A and 3B). The excitation is assumed to be uniform in the radial direction (within the wall). In time, apply a square pulse as shown in FIG. 3C; this forcing function approximately represents the acoustic radiation force from a standard ultrasound transducer employed in a typical SWE setup. Full wave simulation with the applied force, tube geometry, and material properties results in the top wall response shown in FIG. 4. Note that in the actual SWE experiments, the tissue responses are recorded at a later time after applying the acoustic radiation push. Considering this, consider the response after 0.78 ms; in fact, the time axis in FIG. 4 is shifted by 0.78 ms.

### INVERSION FRAMEWORK

### Approach 1

The idea in this approach is to minimize the difference between the measured and simulated phase velocity dispersion. The multimodal framework in which the measured phase velocity is specifically matched with the simulated phase velocity corresponding to the two circumferential modes. The objective function in the inversion analysis takes the form,

$${F}_{objective} = \frac{{\sum }_{{f}_{i} = {f}_{1}}^{{f}_{2}} {( {c}_{2}^{s} ( {f}_{i} , q ) - {c}^{m} ( {f}_{i} ) )}^{2} + {\sum }_{{f}_{i} = {f}_{3}}^{{f}_{4}} {( {c}_{1}^{s} ( {f}_{i} , q ) - {c}^{m} ( {f}_{i} ) )}^{2}}{{\sum }_{{f}_{i} = {f}_{1}}^{{f}_{2}} {( {c}^{m} ( {f}_{i} ) )}^{2} + {\sum }_{{f}_{i} = {f}_{3}}^{{f}_{4}} {( {c}^{m} ( {f}_{i} ) )}^{2}} , q = [ {G}_{0} , ξ ] , \qquad ( 17 )$$

where, c₁^{s} and c₂^{s} are the simulated phase velocities corresponding to the modes 1 and 2, respectively, and c^{m} is the phase velocity from the measured data. Based on experimental observations, mode matching happens for, f₁=400 Hz, f₂=600 Hz, f₃=800 Hz, and f₄=1200 Hz. These specific frequency windows may change with the given data as they depend on the geometric and material properties of the tube. The inversion parameters are, q=[G₀,ξ], where G₀ is the elasticity measure and ξ represents the attenuation measure (e.g. for Kelvin-Voigt model, it is relaxation time, τ and for Spring-pot model, it is fractional order, α).

To compute the measured dispersion, first transform the spatiotemporal (x-t) data to the Fourier space (k-ω) by applying 2D Fast Fourier transformation. Then pick the peak values in the Fourier space and plot the phase velocity, cₚ=ω/k as a function of cyclic frequency (Hz). For the simulated dispersion, the steps are mentioned in the dispersion relation through k(ω) analysis section above.

With respect to inversion, the gradient-based optimization works well in this context, but other methods could also serve this purpose. The interior point algorithm was employed with the BFGS Hessian approximation (MATLAB fmincon function), with box constraints at ±30% of the mean value. Given the small parameter space, gradients were computed using the finite difference technique. More details on this approach can be found in “Multimodal guided wave inversion for arterial stiffness: methodology and validation in phantoms” by Roy et al. (Phys. Med. Biol. 66 115020 (2021)).

### Approach 2

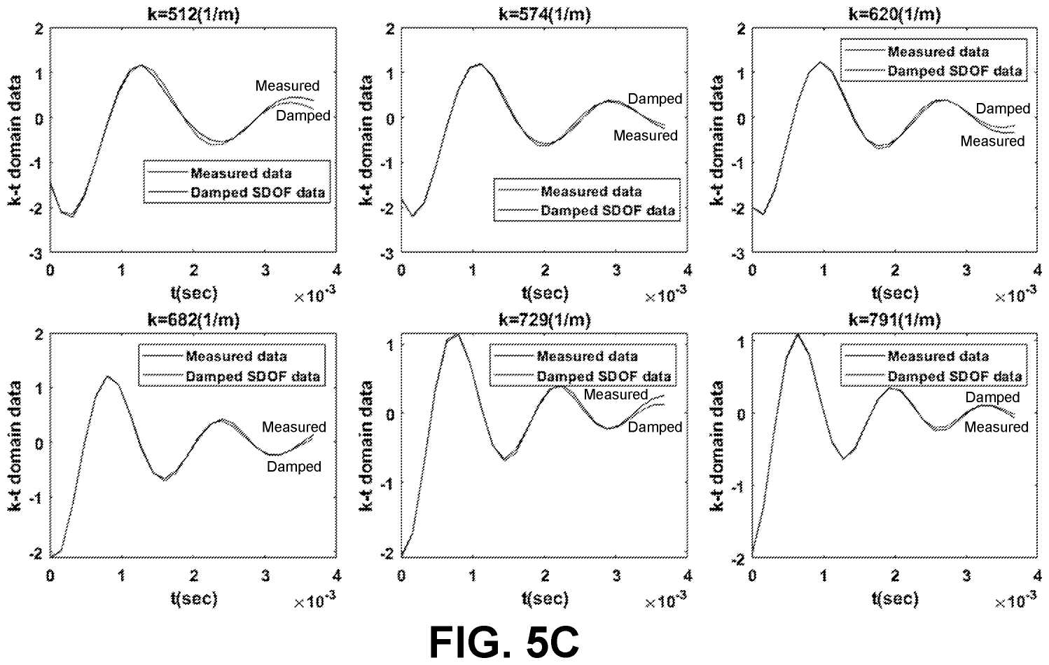

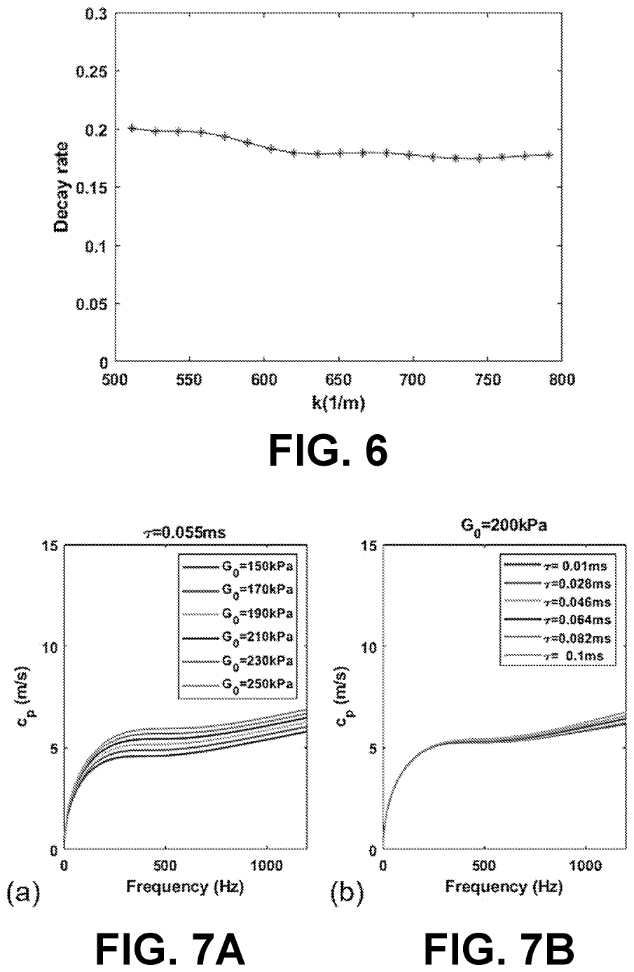

The idea here is to look at the decay rate of the wavenumber-time (k-t) domain response; this is based on the hypothesis that response for a given k at the top of the artery can be approximated as a single-degree-of-freedom system. The steps are thus, (1) transforming the entire x-t (including both left and right propagating parts) data to the k-t domain data through Fast Fourier Transform along the axial direction x, (2) for each of the wavenumbers ranging from 500 to 800 (1/m) (the range of k depends on various parameters including, but not limited to, geometry, forcing function, and the ultrasound data), compare it with a damped Single Degree of Freedom (SDOF) system to estimate the decay rate. These steps are highlighted in FIGS. 5A-5C. FIG. 5A illustrates the full measured data and FIG. 5B illustrates the corresponding k-t domain data. FIG. 5C illustrates the finally matching the k-t domain data with the damped SDOF system at specific k ranges. In FIG. 6, the final result is plotted as the estimated decay rate as a function of wavenumber.

With respect to the second step of estimating the decay rate, consider the gradient-based optimization to minimize the difference between the considered k-t domain data and the damped SDOF responses. For this minimization, MATLAB's unconstrained optimization function, fminunc, was employed.

### Approach 3

In this approach, the full-wave simulation framework can be directly utilized to estimate the viscoelasticity, by maximizing the correlation between measured and simulated wall velocity in x-t domain. In addition, to focus more on higher frequency content and to correct for any bias-type of noise, subtract both measured and simulated (spatiotemporal) data by its mean. Thus, the objective function is given by,

$$ρ ( {v}^{m} , {v}^{s} ) = \frac{1}{N - 1} {\sum }_{i = 1}^{N} ( \frac{{v}_{i}^{m} - {v}_{μ}^{m}}{{v}_{σ}^{m}} ) ( \frac{{v}_{i}^{s} - {v}_{μ}^{s}}{{v}_{σ}^{s}} ) , \qquad ( 18 )$$

where N is the total number of data points in the x-t domain. The v^{m} and v^{s} are the measured and simulated motions. The mean and standard deviation of the measured velocity are,

$${v}_{μ}^{m} = \frac{1}{N} {\sum }_{i = 1}^{N} {v}_{i}^{m} , {v}_{σ}^{m} = \sqrt{\frac{{\sum }_{i = 1}^{N} {( {v}_{i}^{m} - {v}_{μ}^{m} )}^{2}}{N}} . \qquad ( 19 )$$

Similarly, for the simulated velocity, the mean and standard deviation are,

$${v}_{μ}^{s} = \frac{{\sum }_{i = 1}^{N} {v}_{i}^{s}}{N} , {v}_{σ}^{s} = \sqrt{\frac{{\sum }_{i = 1}^{N} {( {v}_{i}^{s} - {v}_{μ}^{s} )}^{2}}{N}} . \qquad ( 20 )$$

### Approach 4

In this approach, match the measured and simulated data in wavenumber-frequency (k-ω) domain. To get the (k-ω) domain response of the measured data, 2D FFT is applied in the acquired space-time motion data. For the simulated response, utilize the full-wave simulation framework which computes the response in (k-ω) domain in an intermediate step (see para. [0026] for details). In the inversion framework, similar to Approach 3, use the correlation based objective function,

$$ρ ( {V}^{m} , {V}^{s} ) = \frac{1}{N - 1} {\sum }_{i = 1}^{N} ( \frac{{V}_{i}^{m} - {V}_{μ}^{m}}{{V}_{σ}^{m}} ) ( \frac{{V}_{i}^{s} - {V}_{μ}^{s}}{{V}_{σ}^{s}} ) , \qquad ( 21 )$$

where, V is the velocity data in (k,ω) domain. All the subscripts and superscripts are defined in the previous approach.

### Other Approaches

While it is advocated for correlating the motion in space-time or wavenumber-frequency domain, depending on experimental data, it may be beneficial to explore the correlation of measured and simulation data in other domains, i.e., wavenumber-time, space-frequency domains. These approaches are not explicitly validated here but can be considered as valuable alternatives in the approach.

### SENSITIVITY STUDY

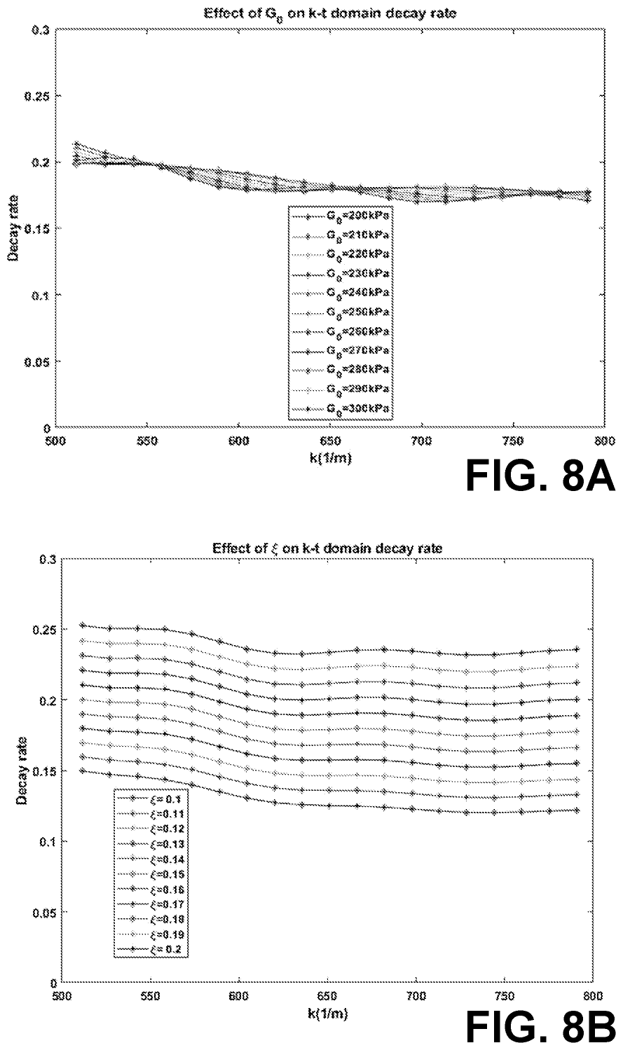

In this section, the sensitivity of the two parameters namely the elastic modulus G₀ and the relaxation time τ is examined for all four approaches mentioned in the previous section. Sensitivity analysis was performed through perturbing one parameter at a time. FIGS. 7A and 7B show the sensitivity of the parameters for the Approach 1. FIG. 7A illustrates the effect of the elastic modulus keeping the relaxation time constant at 0.055 ms, and FIG. 7B illustrates the effect of the relaxation time keeping the elastic modulus at 200 kPa on the phase velocity dispersion with Approach 1, in accordance with various embodiments of the present disclosure. As clearly observed in FIGS. 7A and 7B, the only influential parameter is the elastic modulus, G₀. Therefore, this Approach is well suited for the estimation of the elastic modulus, but not the attenuation.

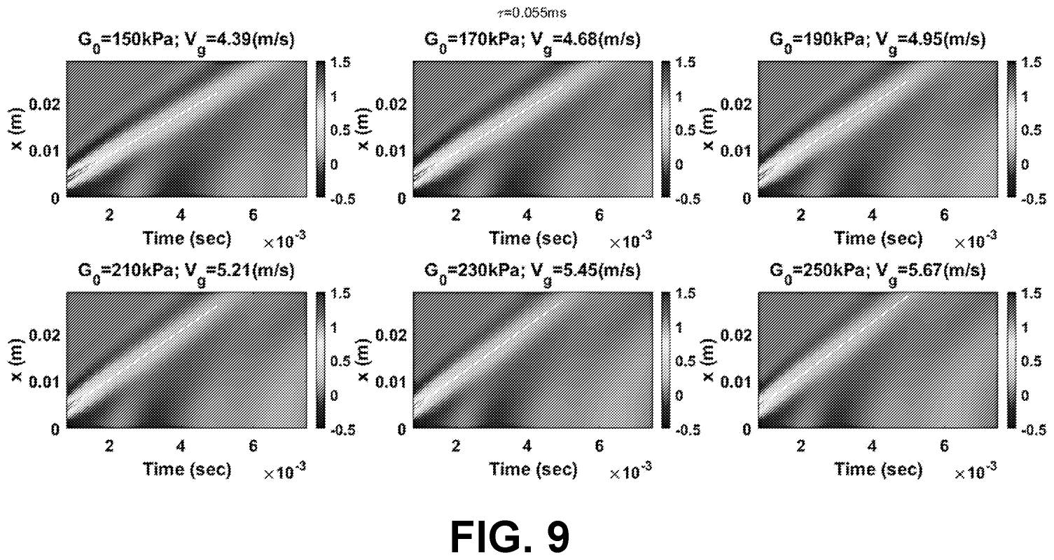

Sensitivity study results for Approach 2 are presented in FIGS. 8A and 8B. FIG. 8A illustrates the effect of elastic modulus maintaining the damping ratio at 0.15, and FIG. 8B illustrates the effect of damping ratio maintaining the elastic modulus at 250 kPa, on the k-t domain decay rate. The influential parameter is the damping ratio ξ but not the modulus. Therefore, this Approach can be considered to estimate viscoelasticity if there is an alternative way to obtain the modulus.

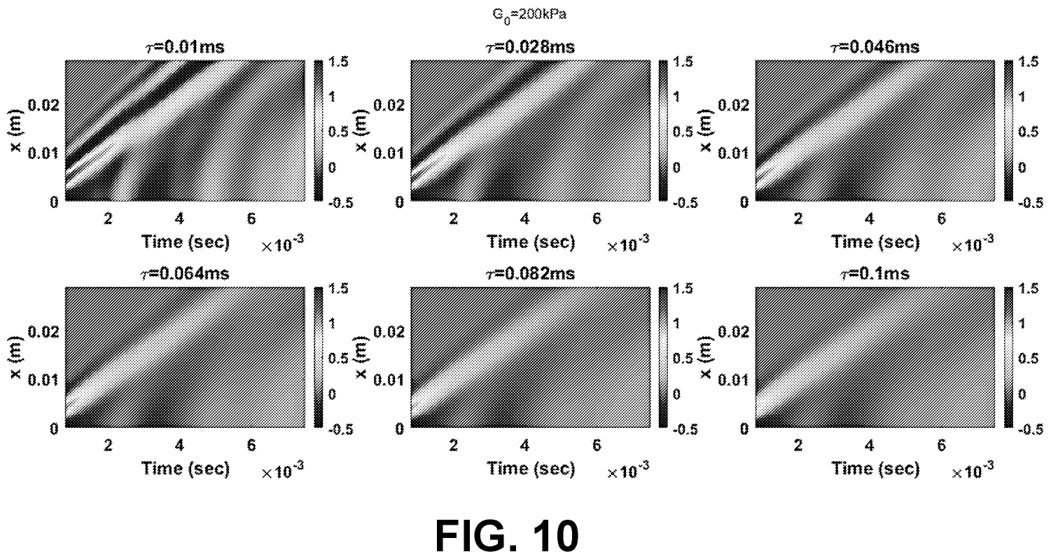

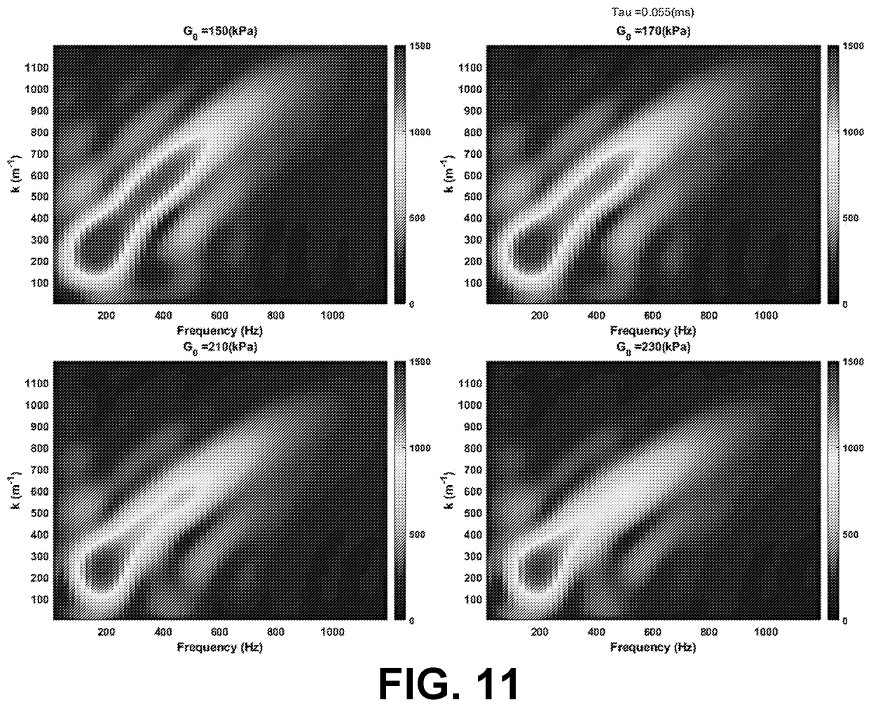

For Approach 3, both parameters are sensitive as shown in FIGS. 9 and 10, which show the effect of modulus and relaxation time. FIG. 9 illustrates the effect of modulus keeping the relaxation time at 0.055 ms and FIG. 10 illustrates the effect of relaxation time keeping the modulus parameter of 200 kPa. In FIG. 9, the slope of the pulse (which represents group velocity of the propagating wave) in space-time domain changes, while in FIG. 10, the decay rate along the direction of propagation is altered.

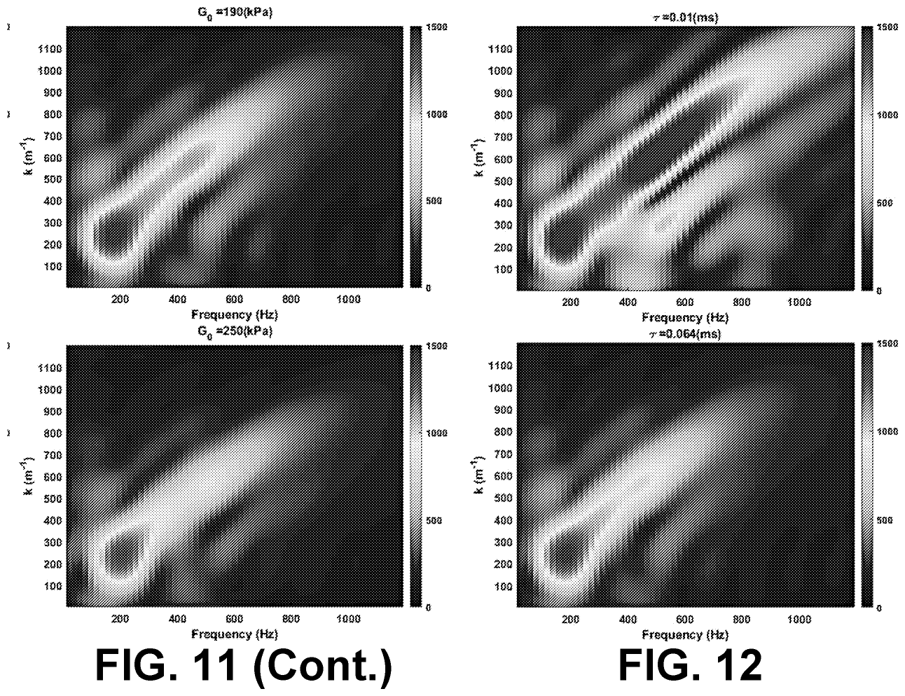

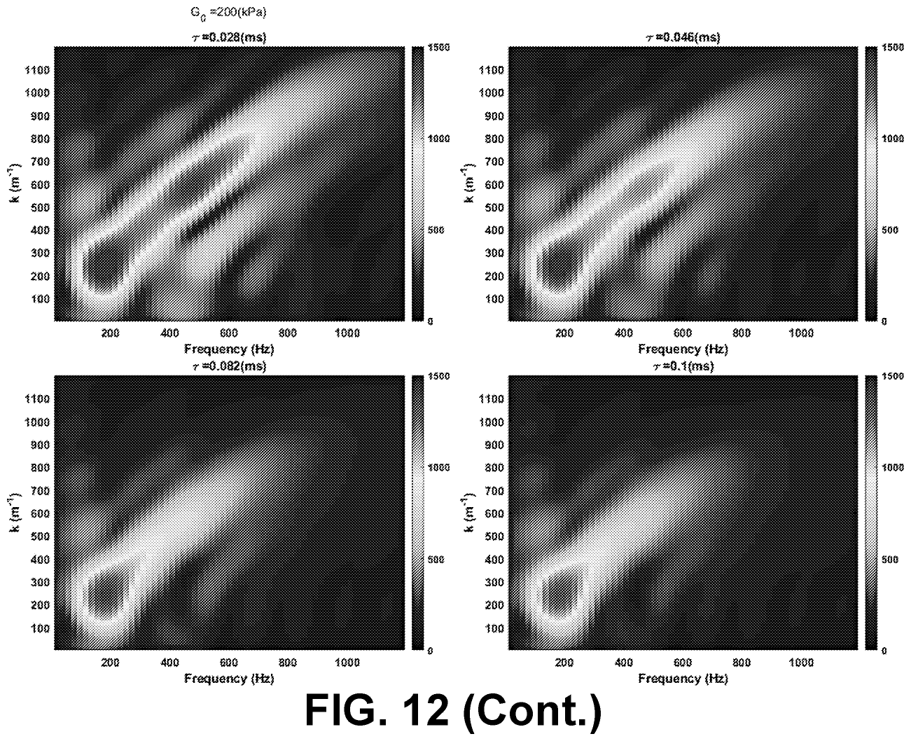

For Approach 4, the sensitivities to modulus and relaxation time are shown in FIGS. 11 and 12, respectively. As observed, both modulus and relaxation time parameters are influential, indicating that Approach 4 can itself be considered to estimate these two parameters.

### PROPOSED APPROACH

Given the modulus parameter is the only influential parameter in Approach 1, it is recommended for the modulus inversion. In fact, satisfactory results were observed for the modulus estimation with Approach 1 not only with synthetic data, but also with real experimental data (see “Multimodal guided wave inversion for arterial stiffness: methodology and validation in phantoms” by Roy et al. (Phys. Med. Biol. 66 115020 (2021)) for details). As for Approach 2, only the attenuation is the influential parameter; thus, this approach can potentially be used to invert for viscosity, after estimating elastic modulus using Approach 1. From FIGS. 8A and 8B, the inverted decay rate (0.18-0.20) was obtained.

In Approaches 3 and 4, both parameters are influential and thus these approaches can be used by itself to invert for viscoelasticity.

Given the confidence gained through validation of Approach 1, a two-step process is proposed: (1) evaluate the elastic modulus from the phase velocity dispersion (Approach 1), then (2) estimate complete viscoelastic parameters from the correlation between the measured velocity and the full wave data in the k-ω domain (Approach 4). While Approach 3 can also be used in the second part, Approach 4 is recommended due to increased convexity in the objective function, which is helpful for inverting with noisy data. It further leads to faster convergence of the optimization algorithm. Verification of Approaches 3 and 4 was carried out using synthetic data, for two different models of viscoelasticity.

### Verification of Approach 3 Using Synthetic Data

To examine the effectiveness of Approach 3, synthetic data was first generated from

the full-wave simulation (see para. [0026]), polluted with bias and both additive noise and multiplicative noise:

$${v}_{synthetic} = {v}_{full - wave} ( 1 + {ò}_{multiplicative} ) + {ò}_{additive} + b . \qquad ( 22 )$$

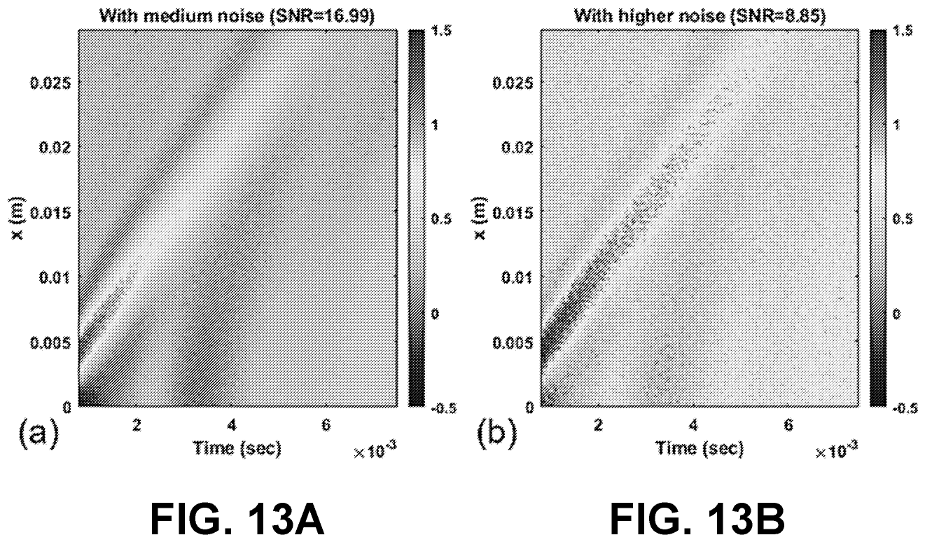

Two different levels of noise were considered. For the medium noise level, the bias value, b is 0.01 and the noise distributions are taken as Gaussian: ò_{multiplicative}˜N(μ=0, σ²=0.07²) and ò_{additive}˜N(μ=0, σ²=0.001²). For higher level of noise, the bias value b is chosen as 0.02, and the noise distributions are ò_{multiplicative}˜N(μ=0, σ²=0.10²) and ò_{additive}˜N(μ=0, σ²=0.003²). Representative synthetic data with noise added for these two cases are shown in FIGS. 13A and 13B, using a modulus of 200 kPa and relaxation time of 0.055 ms.

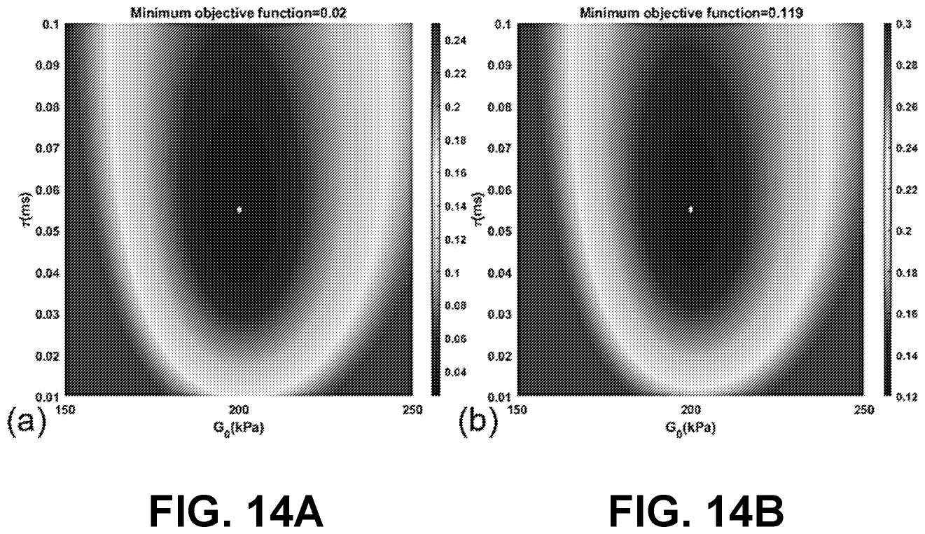

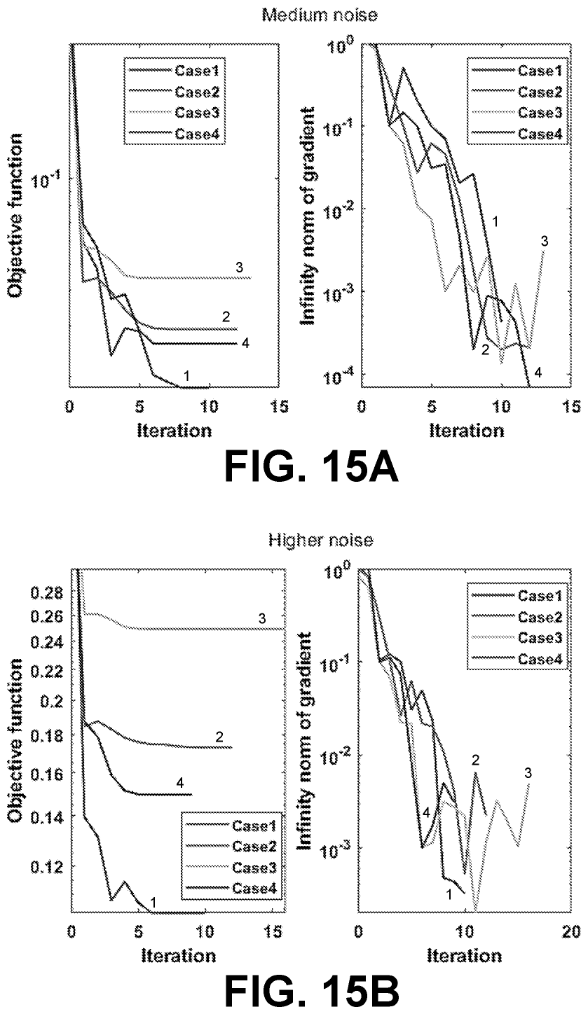

Approach 3 was applied to the two polluted synthetic data sets from a Kelvin-Voigt viscoelastic model (see FIGS. 13A and 13B). Specifically, examine the objective function in equation (18) between synthetic, polluted data and the predicted data from full-wave simulations for several elastic modulus and the relaxation time values. FIGS. 14A and 14B show the variation of the objective function over the considered parameter space for Kelvin-Voigt viscoelastic model for synthetic data with medium noise and high noise, respectively. As observed, for both polluted data sets, the minimum point at the expected location is obtained. In addition, from FIGS. 14A and 14B, it can be observed that the objective function is well behaving and convex near the optimal point. Encouraged by this, gradient optimization (interior point algorithm with BFGS Hessian) can be utilized for various sets of synthetic data with different combination of parameter values. In all these experiments, as shown in table 1, the results converge to a point near the expected values, indicating the effectiveness of the proposed approach. The iteration histories of the gradient optimization for Kelvin-Voigt viscoelastic model for synthetic data with (a) medium noise and (b) higher noise are presented in FIGS. 15A and 15B.

TABLE 1
Gradient-based optimization results for Kelvin-Voigt model.

| | Actual | | Inverted parameters | | | | | | | |
|---|---|---|---|---|---|---|---|---|---|---|
| | parameters | | Medium noise | | Higher noise | | Percentage difference | | | |
| | G₀ | τ | G₀ | τ | G₀ | τ | Medium noise | | Higher noise | |
| Case no. | (kPa) | (ms) | (kPa) | (ms) | (kPa) | (ms) | G₀ | τ | G₀ | τ |
| 1 | 150 | 0.03 | 150.05 | 0.0301 | 150.06 | 0.0307 | 0.030 | 0.267 | 0.043 | 2.233 |
| 2 | 150 | 0.08 | 149.997 | 0.0801 | 149.97 | 0.0798 | 0.002 | 0.063 | 0.019 | 0.263 |
| 3 | 250 | 0.08 | 249.76 | 0.0797 | 250.45 | 0.0803 | 0.096 | 0.438 | 0.179 | 0.350 |
| 4 | 250 | 0.03 | 250.10 | 0.0300 | 249.85 | 0.0300 | 0.040 | 0.033 | 0.059 | 0.100 |

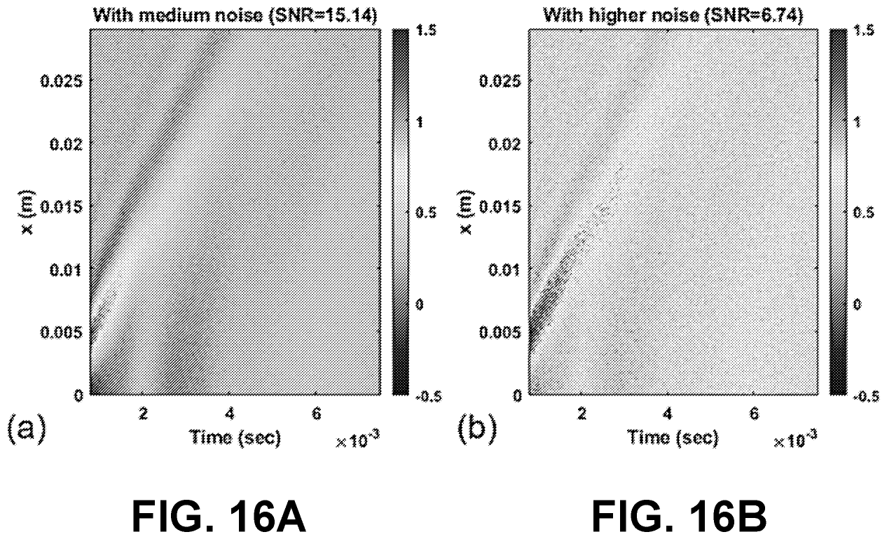

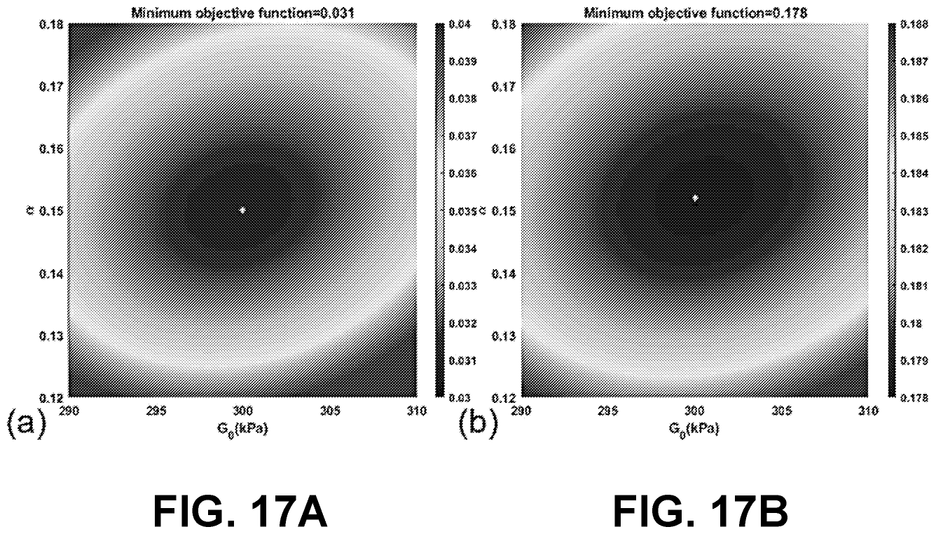

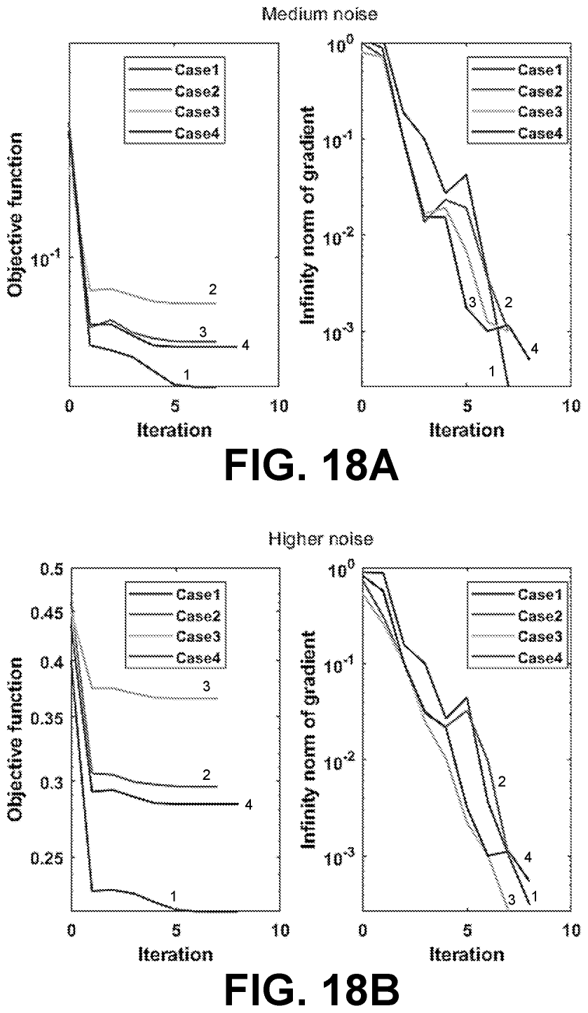

The above process was repeated for the spring-pot viscoelastic model. The corresponding noise-laden synthetic data are shown in FIGS. 16A and 16B corresponding to medium and higher noise cases. Examples of objective function variation for the two noise cases are shown in FIGS. 17A and 17B, which again indicates a well-behaving and locally convex objective function. Similar to the previous example, here also, perform the gradient-based optimization to invert for both modulus, G₀ and the fractional order, α. The inversion results are shown in Table 2, which indicates the effectiveness of the proposed approach for more complicated spring-pot viscoelastic model. FIGS. 18A and 18B show the iteration histories of the gradient optimization for spring-pot viscoelastic model for synthetic data with medium noise and higher noise.

TABLE 2
Gradient optimization results for the spring-pot model.

| | Actual | | Inverted parameters | | | | | | | |
|---|---|---|---|---|---|---|---|---|---|---|
| | parameters | | Medium noise | | Higher noise | | Percentage difference | | | |
| | G₀ | | G₀ | | G₀ | | Medium noise | | Higher noise | |
| Case no. | (kPa) | α | (kPa) | α | (kPa) | α | G₀ | α | G₀ | α |
| 1 | 250 | 0.12 | 250.10 | 0.1209 | 249.82 | 0.1202 | 0.040 | 0.750 | 0.071 | 0.150 |
| 2 | 250 | 0.18 | 249.67 | 0.1790 | 249.67 | 0.1780 | 0.132 | 0.578 | 0.133 | 1.139 |
| 3 | 350 | 0.18 | 349.63 | 0.1776 | 349.36 | 0.1797 | 0.106 | 1.350 | 0.184 | 0.172 |
| 4 | 350 | 0.12 | 350.02 | 0.1207 | 350.16 | 0.1208 | 0.005 | 0.608 | 0.044 | 0.692 |

### Verification of Approach 4 Using Synthetic Data

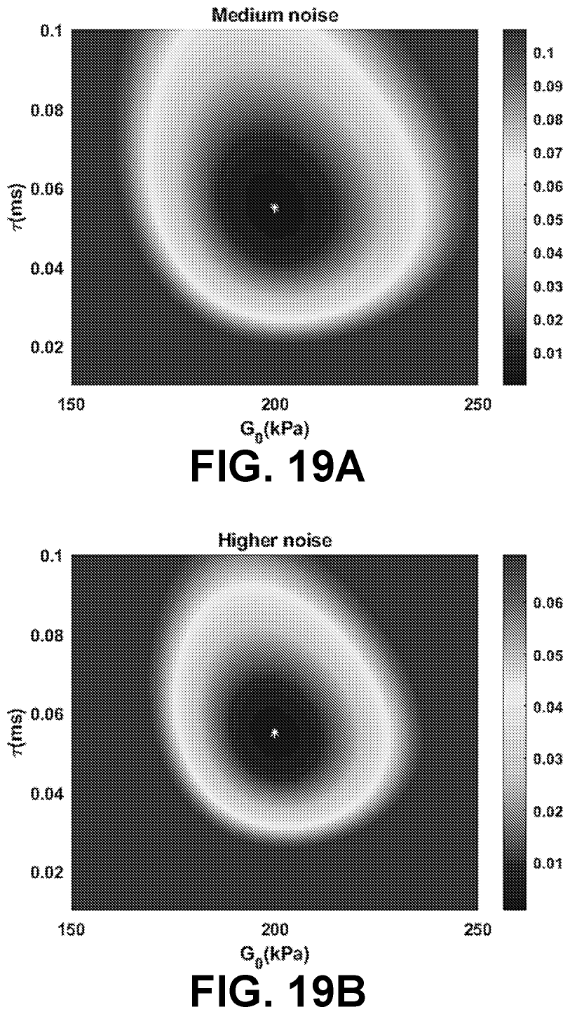

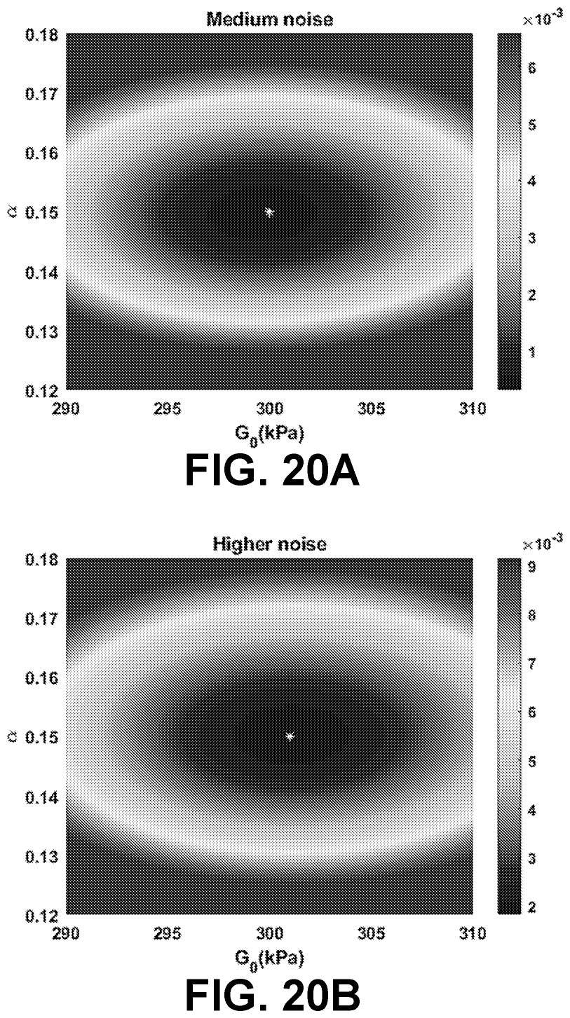

The same synthetic data used for Approach 3 is applied to demonstrate the effectiveness of Approach 4. FIGS. 19A-19B and 20A-20B show the variation of the objective function (equation (21)) corresponding to medium and higher noise cases for Kelvin-Voigt and spring-pot viscoelastic model respectively. The objective function is more convex than the previous case indicating the faster convergence in the optimization step.

### SHEAR MODULUS ESTIMATION

Ultrasound-based shear wave elastography (SWE) has shown promise for noninvasively estimating tissue stiffness by using a focused acoustic radiation pulse to induce transient motion in tissue, subsequently imaging the velocity at which transverse waves propagate away from the region of excitation. In bulk tissue, where the wavelength of the outwardly propagating wave is much smaller than the tissue dimensions, this velocity can be related to the shear modulus by G=ρC² where ρ is tissue density and C is the wave velocity. However, accurately measuring arterial stiffness in vivo with this technique has proven difficult. the geometric properties of arteries cause waves of different frequencies to propagate at different velocities (i.e., C=C(ω)), a phenomenon known as dispersion. Present clinical implementations of SWE measure the group velocity (C_{g}) of the induced wave, which is strongly dependent upon arterial geometry. Failing to account for this can lead to significant geometry-dependent underestimation of arterial shear modulus.

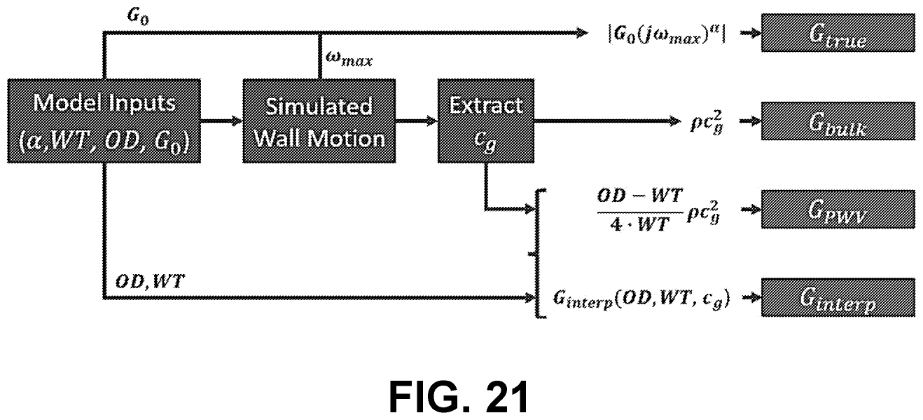

To address this, a novel method of producing a minimally-biased estimate of vascular shear modulus is proposed by considering local lumen radius (r), wall thickness (h) and induced wave group velocity (C_{g}), all of which are quantities available from commercial SWE-equipped ultrasound scanners. First, a validated semi-analytical finite element (SAFE) model can be used to simulate induced wave motion on the proximal wall of an artery after insonification with a focused acoustic radiation force pulse. This model can incorporate the beam shape used to generate the acoustic radiation force excitation. This permits in silico variation of tissue properties including h, r, and viscoelastic modulus parameters. For example, for a spring-pot model, the modulus parameters would be G₀ and α, where the frequency-dependent modulus is given by, G(ω)=G₀(jω)^{α}. Another alternative would be to use Kelvin-Voigt shear modulus model G(ω)=G₀+jωη, where G₀ and η would be the modulus parameters. The SAFE model can also allow the simulation of other types of viscoelasticity as well. Once the space-time response is computed, a standard time-to-peak, Radon transform, or other algorithm can be used to obtain the C_{g} of the induced wave, which is the quantity that a commercial ultrasound scanner would measure. Other alternative methods could be used for measurement of the group velocity such as cross-correlation. FIG. 21 illustrates an example of a process for performing shear modulus estimation using the proposed technique.

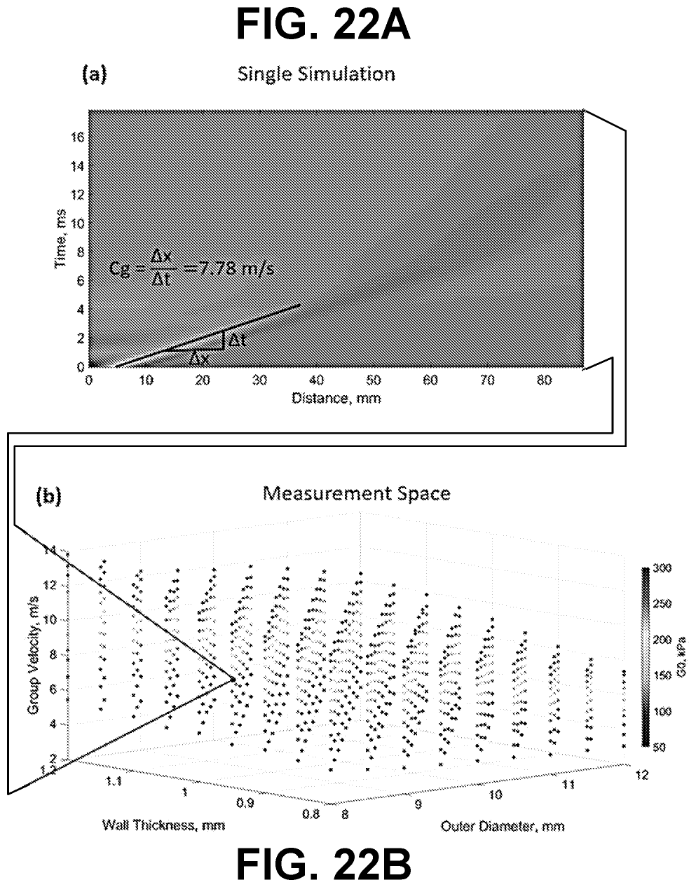

By repeating this simulation across a set of vessel radii (r_{grid}), wall thicknesses (h_{grid}), and shear moduli (G_{grid}) for a fixed α (assuming a spring-pot model), a numerical matrix G(r,h,C_{g}) can be obtained, where r∈r_{grid}, h∈h_{grid}, and G∈G_{grid}. The coordinates of this three-dimensional matrix correspond to measured quantities that are available via clinical ultrasound scanners, and the value of the matrix at any given coordinate is the low-frequency shear modulus of the tissue that would have produced those measured quantities. FIG. 22A illustrates an example of the SAFE simulation for a single value of outer diameter, wall thickness, and underlying shear modulus. The group velocity of the traveling wave is measured using a time-to-peak algorithm. FIG. 22B illustrates an example of the interpolation matrix populated with simulation results. Each coordinate corresponds to a particular set of simulated measurements (wall thickness, outer diameter, and group velocity). The value of the matrix at any coordinate is the shear modulus corresponding to that measurement set.

As C_{g} values are measured in the experiments, they are not guaranteed to fall on a particular pre-defined regular grid. Nonetheless, they can be mapped to a uniform grid via interpolation (e.g., using the MATLAB scatteredinterpolant function). Alternatively, they can be accommodated by initializing C_{grid} to a fine resolution (e.g., 0.01 m/s increments) such that the Euclidean distance between the (r,h,C_{g}) coordinates corresponding to a given simulation and the nearest available grid point in measurement space is negligible. Initially, only those coordinates corresponding to simulations are initialized with numerical values, while others are initialized as “NaN.” Missing values can be filled in via interpolation.

The resulting fully-populated numerical matrix directly is a “look-up table” for G₀ values corresponding to a given set of arterial measurements of C_{g}, provided that those measurements fall on one of the defined grid points. G₀ values for off-grid measurements (e.g., in cases where r∈r_{grid}, h∈h_{grid}, and/or C_{g}∈C_{grid}) can be estimated via 3D interpolation on the populated matrix, provided that all parameters fall between the minimum and maximum parameter values contained within the interpolation matrix. Using this interpolation-based technique yields estimates of G₀ that exhibit substantially less bias and reduced dependence on geometry as compared to conventional techniques that consider only C_{g} and neglect geometry.

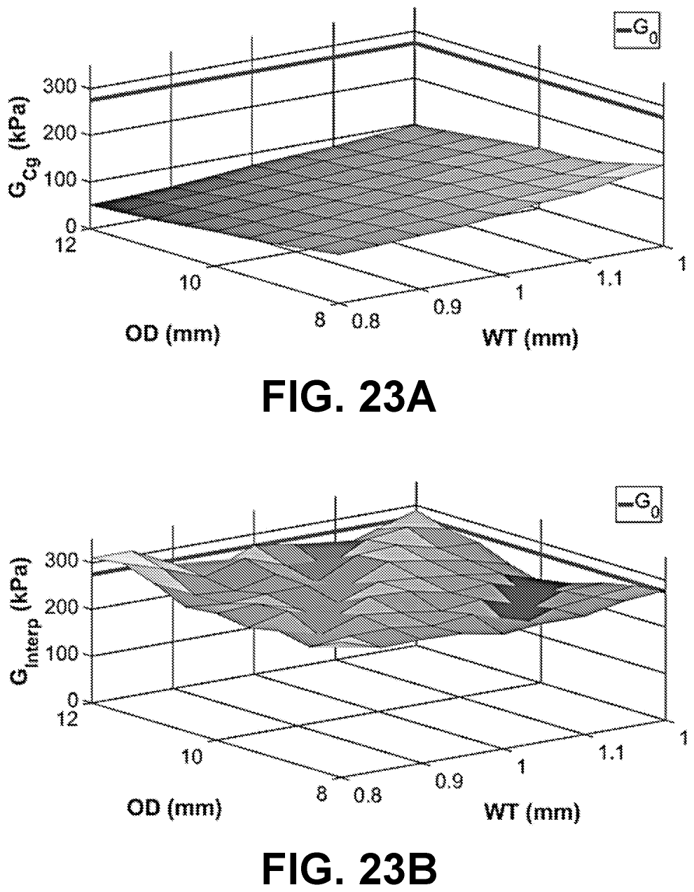

FIG. 23A illustrates an example of geometry-dependent shear modulus underestimation introduced by using the bulk wave approximation G=ρ for a range of outer diameters and wall thicknesses at a given underlying shear modulus of G₀=275 kPa. Failing to incorporate geometry causes many measured values of C_{g} to map to the same value of G₀ (and vice-versa). As illustrated in FIG. 23B, these effects can be ameliorated using the proposed simulation and interpolation-based approach.

This technique may also be extended to accommodate an unknown viscosity parameter by measuring both the group velocity G₀ and a secondary parameter related to viscosity, such as the decay rate of the peak amplitude of the propagating wave with respect to space. Exploiting both measurements can permit simultaneous estimation of both modulus parameters, e.g., α and G₀ in the context of spring-pot model, or G₀ and η in the context of Kelvin-Voigt model. Alternatively, this technique can also consider a purely elastic material by setting the viscosity parameter to 0 during the generation of measurement space.

Additional modeling can incorporate surrounding tissue, but preliminary studies indicate this may not be necessary. Different look-up tables can be constructed depending on the acoustic radiation force profiles that might be applied for generating waves in arterial or venous vessels, but this can be initially evaluated and stored in the ultrasound scanner for rapid evaluations.

This method can be implemented as a software addition to SWE-equipped scanners. In addition to the group velocity measured using standard SWE, this technique utilizes inputs of vessel radius and wall thickness which can be measured with the same ultrasound equipment and automatically or semi-automatically extracted as part of the SWE measurement. The technique can provide a more unique and comprehensive estimate of arterial vessel mechanical properties compared to conventional methods by incorporating the complicated physics of wave propagation in cylindrical arteries.

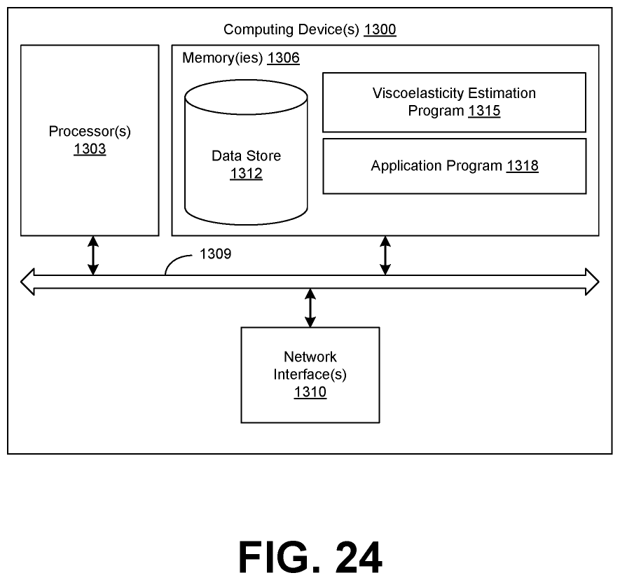

With reference to FIG. 24, shown is a schematic block diagram of a computing (or processing) device 1300 that can be utilized for similarity and compatibility checking and management for modular construction using the described techniques. In some embodiments, among others, the computing device 1300 may represent a mobile device (e.g., a smartphone, tablet, computer, etc.) or other processing device. Each computing device 1300 includes processing circuitry comprising at least one processor circuit, for example, having a processor 1303 and a memory 1306, both of which are coupled to a local interface 1309. To this end, each computing device 1300 may comprise, for example, at least one server computer or like device. The local interface 1309 may comprise, for example, a data bus with an accompanying address/control bus or other bus structure as can be appreciated.

In some embodiments, the computing device 1300 can include one or more network interfaces 1310. The network interface 1310 may comprise, for example, a wireless transmitter, a wireless transceiver, and a wireless receiver. As discussed above, the network interface 1310 can communicate to a remote computing device using a Bluetooth protocol. As one skilled in the art can appreciate, other wireless protocols may be used in the various embodiments of the present disclosure.

Stored in the memory 1306 are both data and several components that are executable by the processor 1303. In particular, stored in the memory 1306 and executable by the processor 1303 are viscoelasticity estimation program 1315, application program 1318, and potentially other applications. Also stored in the memory 1306 may be a data store 1312 and other data. In addition, an operating system may be stored in the memory 1306 and executable by the processor 1303.

It is understood that there may be other applications that are stored in the memory 1306 and are executable by the processor 1303 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

A number of software components are stored in the memory 1306 and are

executable by the processor 1303. In this respect, the term “executable” means a program file that is in a form that can ultimately be run by the processor 1303. Examples of executable programs may be, for example, a compiled program that can be translated into machine code in a format that can be loaded into a random access portion of the memory 1306 and run by the processor 1303, source code that may be expressed in proper format such as object code that is capable of being loaded into a random access portion of the memory 1306 and executed by the processor 1303, or source code that may be interpreted by another executable program to generate instructions in a random access portion of the memory 1306 to be executed by the processor 1303, etc. An executable program may be stored in any portion or component of the memory 1306 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 1306 is defined herein as including both volatile and nonvolatile memory and data storage components. Volatile components are those that do not retain data values upon loss of power. Nonvolatile components are those that retain data upon a loss of power. Thus, the memory 1306 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 1303 may represent multiple processors 1303 and/or multiple processor cores and the memory 1306 may represent multiple memories 1306 that operate in parallel processing circuits, respectively. In such a case, the local interface 1309 may be an appropriate network that facilitates communication between any two of the multiple processors 1303, between any processor 1303 and any of the memories 1306, or between any two of the memories 1306, etc. The local interface 1309 may comprise additional systems designed to coordinate this communication, including, for example, performing load balancing. The processor 1303 may be of electrical or of some other available construction.

Although the viscoelasticity estimation program 1315 and the application program 1318, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the viscoelasticity estimation program 1315 and the application program 1318, that comprises software or code can be embodied in any non-transitory computer-readable medium for use by or in connection with an instruction execution system such as, for example, a processor 1303 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the viscoelasticity estimation program 1315 and the application program 1318, may be implemented and structured in a variety of ways. For example, one or more applications described may be implemented as modules or components of a single application. For example, separate applications can be executed for the viscoelasticity estimation workflows. Further, one or more applications described herein may be executed in shared or separate computing devices or a combination thereof. For example, a plurality of the applications described herein may execute in the same computing device 1300, or in multiple computing devices in the same computing environment. Additionally, it is understood that terms such as “application,” “service,” “system,” “engine,” “module,” and so on may be interchangeable and are not intended to be limiting.

In this work, four approaches to back-calculate the viscoelastic parameters were examined. The examined approaches perform the analysis in three different domains: Approach 1 attempts to match the dispersion curves effectively in wavenumber-frequency domain; Approach 2 matches the decay rate in wavenumber-time domain; and Approach 3 and Approach 4 maximize the correlation of measured and simulated responses in space-time and wavenumber-frequency domains respectively. Through sensitivity analyses, it was observed that Approach 1 works well for estimating the elastic modulus but is not sensitive for the damping parameter. On the other hand, Approach 2 is sensitive to the viscous parameter, but not to the elastic modulus. Hence, a sequential application of these two approaches would lead to viscoelastic inversion. Alternative are Approaches 3 and 4, where both elastic modulus and viscosity parameter are shown to be influential parameters. In fact, the effectiveness of Approaches 3 and 4 is illustrated with the help of noise laden synthetic data. Combining these approaches, e.g., using estimated modulus from Approach 1 and/or estimated viscous parameter from Approach 2 as starting points for estimating both the parameters using Approach 3 or Approach 4 can provide improvements of computational cost.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for estimation of viscoelasticity of an arterial or venous wall, comprising:
obtaining ultrasound data of an arterial or venous wall for a defined acoustic radiation force (ARF); and
determining viscoelasticity of the arterial or venous wall, where the viscoelasticity is determined:
based upon correlation between measured and simulated wall velocity of the ultrasound data in a space-time, a wavenumber-frequency, wavenumber-time, or space-frequency domain, where the simulated velocity is determined for a wall thickness and viscoelastic modulus of the arterial or venous wall through full wave analysis; or
in a sequential manner by determining the elastic part of the modulus by matching measured and simulated group or phase velocities and determining the viscous part by matching measured and simulated decay characteristics; or
using a combination of both.

2. The method of claim 1, wherein determination of the viscoelastic modulus is based upon maximization of a correlation coefficient to within a defined threshold, in the space-time, wavenumber-frequency, wavenumber-time, or space-frequency domain.

3. The method of claim 1, comprising matching measured phase velocity of the ultrasound data with simulated phase velocity corresponding to wave modes of the arterial or venous wall.

4. The method of claim 3, wherein the measured phase velocity is matched with simulated phase velocity to within a defined threshold.

5. The method of claim 3, wherein the wave modes comprise first and second circumferential modes.

6. The method of claim 1, wherein a measured decay profile is matched with simulated decay profile to within a defined threshold.

7. The method of claim 1, comprising determining shear modulus prior to viscoelasticity determination based upon shear wave elastography (SWE) measurements.

8. The method of claim 7, wherein the SWE measurements comprise local lumen radius (r), wall thickness (h) and induced wave group velocity (C_{g}).

9. The method of claim 7, wherein the shear modulus is determined using an interpolation matrix.

10. The method of claim 9, wherein the interpolation matrix is generated based at least in part upon acoustic radiation force push spatial and temporal characteristics.

11. The method of claim 10, wherein the interpolation matrix is generated based upon local lumen radius (r), wall thickness (h) and induced wave group velocity (C_{g}).

12. A system for estimation of viscoelasticity of an arterial or venous wall, comprising:
an ultrasound scanner configured for shear wave elastography (SWE); and
a computing device comprising a processor and memory, the computing device configured to at least:
obtain ultrasound data of an arterial or venous wall for a defined acoustic radiation force (ARF), the ultrasound data obtained via the ultrasound scanner; and
determine viscoelasticity of the arterial or venous wall, where the viscoelasticity is determined:
based upon correlation between measured and simulated wall velocity of the ultrasound data in a space-time, a wavenumber-frequency, wavenumber-time, or space-frequency domain, where the simulated velocity is determined for a wall thickness and viscoelastic modulus of the arterial or venous wall through full wave analysis; or
in a sequential manner by determining the elastic part of the modulus by matching measured and simulated group or phase velocities and determining the viscoelasticity part by matching measured and simulated wall velocities; or
using a combination of both.

13. The system of claim 12, wherein determination of the viscoelastic modulus is based upon maximization of a correlation coefficient to within a defined threshold, in the space-time, wavenumber-frequency, wavenumber-time, or space-frequency domain.

14. The system of claim 12, wherein matching measured and simulated phase velocities comprises matching measured phase velocity of the ultrasound data with simulated phase velocity corresponding to wave modes of the arterial or venous wall.

15. The system of claim 14, wherein the wave modes comprise first and second circumferential modes.

16. The system of claim 12, wherein shear modulus is determined prior to viscoelasticity determination based upon shear wave elastography (SWE) measurements.

17. The system of claim 16, wherein the SWE measurements comprise local lumen radius (r), wall thickness (h) and induced wave group velocity (C_{g}).

18. The system of claim 17, wherein shear modulus is determined using an interpolation matrix generated based at least in part upon acoustic radiation force push spatial and temporal characteristics.

19. The system of claim 12, wherein the ultrasound data is obtained from the ultrasound scanner in real time or near real time.

20. The system of claim 12, wherein the ultrasound data is obtained from memory after recording by the ultrasound scanner.
